(19) United States
(12) Patent Application Publication
(10) Pub. No.: US 2011/0008783 A1
(43) Pub. Date: Jan. 13, 2011

(54) FLUORIMETRIC PROCESS FOR EVALUATING THE INFLUENCE OF A CONDITION ON A BIOLOGICAL SAMPLE, AND APPLICATIONS THEREOF

(76) Inventors: Sylvain Derick, Portet Sur Garonne (FR); Christophe Furger, Toulouse France (FR); Jean-Francois Tocanne, Flourens France (FR)

Correspondence Address: MILES & STOCKBRIDGE PC, 1751 PINNACLE DRIVE, SUITE 500, MCLEAN, VA, 22102-3833, US

(21) Appl. No.: 12/812,005

(22) PCT Filed: Jan. 9, 2009
(86) PCT No.: PCT/EP2009/050235

§ 371 Date: Sep. 10, 2010

(30) Foreign Application Priority Data
Jan. 10, 2008 (FR) 0850136

(51) Int. Cl. C12Q 1/68; G01N 33/557

(52) U.S. Cl. 435/6; 435/7.1

(57) ABSTRACT

The present invention relates to a process for determining the influence of a condition on a biological sample comprising a step consisting in establishing the kinetic profile of the fluorescence emitted, during the excitation at a suitable excitation wavelength, of a fluorescent compound bound to said biological sample, said sample having been, prior to said excitation, subjected to said condition and said process not necessitating the utilization of a fluorescence donor component and of a different fluorescence acceptor component. The present invention also relates to the various applications of such a process.

### TECHNICAL FIELD

The present invention belongs to the technical field of fluorimetric assays applied in biology and more particularly in cell biology.

The present invention relates to a fluorimetric process for evaluating the influence of an experimental and/or environmental condition on a biological sample and proposes applications in the field of in vitro diagnostics, in the field of screening of compounds having a high potential from a therapeutic point of view and in the field of quality control.

### PRIOR ART

The detection of cell death is the subject of many applications particularly in the fields of cancerology and toxicology. The measurement of cell death is generally based on the observation and analysis of major events taking place in the final stages of the life of the cell in particular the changes in the functioning of the mitochondrion, in the organisation and permeability of the plasma membrane, or else in the structure of the DNA.

Various technologies have been developed which in general target the detection of one of these major stages. The detection of the change in the organisation of the plasma membrane is for example handled by the “annexin” method which exploits the capacity of annexin to bind to phosphatidyl serines which appear on the external surface of the plasma membrane on the approach of cell death (“Annexin V affinity assay: a review on an apoptosis detection system based on phosphatidylserine exposure”, Van Engelans et al, Cytometry, 31:1-9, 1998).

Other methods target the alterations and fragmentations of the DNA in the course of cell death and use nuclear markers such as the SYTO (US patent application 2006/099638 A1; Bradford et al, US patent 2006/263844 A1) the fluorescence whereof is stated to vary depending on the state of the nucleic acids (DNA and RNA) of the cell (“Discrimination of DNA and RNA in cells by a vital fluorescent probe: lifetime imaging of SYTO13 in healthy and apoptotic cells”, Van Zandvoort et al, Cytometry, 47:226-235, 2002). The above methods require instruments of the flow cytometry type which remain very expensive, are difficult to use and only allow the processing of samples in the form of cell suspensions.

There is thus a real need to have methods which are easier to implement for detecting apoptogenic conditions leading and/or for identifying compounds capable of modulating apoptosis. This need is real in the context of the high throughput screening carried out by pharmaceutical companies which requires the utilisation of simple and robust tests. The provision of such in vitro methods finally makes it possible to restrict tests on animals, which are costly and not compatible with high throughput screening.

### DISCLOSURE OF THE INVENTION

The present invention makes it possible to provide a solution to the aforesaid technical problems since it discloses a novel fluorimetric process making it possible to characterize the influence of an environmental and/or experimental condition on a biological sample on the basis of the kinetic profile of the fluorescence emitted by a fluorescent compound placed in contact with said biological sample subjected to said environmental and/or experimental condition.

The present invention is based on the work of the inventors which made it possible to perfect a method for analysis of cell death. It exploits the ability of fluorophores binding to DNA to have their fluorescence properties perturbed by this binding.

Without desiring to be bound by any one theory, it appears that fluorophores bound to DNA are located in a confined space which, at a sufficient labelling level, favours an extensive mechanism of energy transfer by resonance between identical molecules leading to partial inhibition of their fluorescent emission (this is referred to as homo-transfer or homo-FRET).

Under continuous illumination of the sample, fluorophores are photodestroyed (“photobleaching”) which results in a progressive removal of the homoFRET process and hence the restoration of the fluorescence emission properties of the fluorophores which are still intact. These latter can then be considered to be “photoactivated”. This “photoactivation” thus makes it possible to measure kinetically the level of restoration of the fluorescence emitted by the sample. The “photoactivation” capacity of fluorophores binding to DNA is closely linked to the distance existing between the various molecules present on the DNA molecule. An example of such molecules has recently been described (“BENA435, a new cell-permeant photo-activated green fluorescent DNA probe”, Erve et al, Nucleic Acids Research, vol. 34, no. 5, 2006).

Moreover, the state of the DNA is known to be modified in many physiological situations, particularly cell death (“Degradation of chromosomal DNA during apoptosis”, Nagata et al, Cell Death and Differentiation, 10:108-116, 2003). The invention is based on the idea that the level of restoration of fluorescence described above will vary depending on the state of the DNA (normal, condensed, degraded, fragmented, . . . ), the different states inducing variable distances between fluorophores, and hence a different state of fluorescence inhibition. This thus results in a variable level of restoration of the fluorescence which is associated with the state of the DNA.

The process of the invention exhibits three major points of interest which differentiate it from the previous methods:
it works on cells in adherent or suspension culture,
the result is quantifiable (establishment of dose-response relationships),
the experimental protocol utilized is extremely simple (addition of the molecule to the culture medium and measurement on standard, inexpensive fluorescence readers).

In addition, the present invention is noteworthy since, on the basis of results obtained in studies connected with apoptosis, it is generalizable to many physiological processes wherein use can be made of the variation in the kinetic fluorescence profile of a fluorescent sample kept under illumination, the different kinetic profiles obtained from different experimental conditions reflecting different states of the sample. The physiological processes capable of being studied by the process of the invention must exhibit two extreme states designated below as state 1 and state 2.

In a state 1, the biosensors situated at a distance compatible with the homo-FRET phenomenon (FRET between identical molecules) emit little observable fluorescence when they are excited (fluorescence inhibition phenomenon). This fluorescence inhibition is then removed under continuous illumination of the sample, which results in an increase in the fluorescence observed. The increase in fluorescence caused by the illumination could result from a phenomenon of photo-degradation of an increasing number of molecules of biosensors. This photodegradation would result in an increase in the distance between non-degraded molecules, a distance which progressively becomes incompatible with the phenomenon of fluorescence inhibition. This progressive removal of the fluorescence inhibition is then revealed by an increase in the fluorescence observed.

In a state 2, the biosensors located at a distance incompatible with the homo-FRET phenomenon are not subjected to the fluorescence inhibition described above. No increase in the fluorescence can be observed on illumination.

Thus, among the physiological processes which can advantageously be studied via the process of the invention, the physiological processes involving the following can be cited:
variations in the state of the structure of the DNA such as a reorganization, degradation or segmentation such as apoptosis, necrosis or cell divisions,
variations in the cell location and/or concentration of compounds such as proteins or nucleic acids such as characteristic mRNAs, such as the interaction between identical or different proteins.

The present invention thus relates to a process for determining the influence of a condition on a biological sample comprising a step consisting in establishing the kinetic profile of the fluorescence emitted, during the excitation at a suitable excitation wavelength, of a fluorescent compound bound to said biological sample, said sample having been, prior to said excitation, subjected to said condition.

It should be noted that the process according to the invention envisages two alternative modes of implementation wherein, in one case, it is the biological sample which is subjected to the condition, whereas the fluorescent compound is already fixed on the sample and, in the other case, it is the sample which is subjected to the condition, before the fluorescent compound is fixed on the sample.

The process of the present invention differs from the prior art particularly by the fact that, on the one hand, it does not require the utilisation of a fluorescence donor component and of a different fluorescence acceptor component as in the pairs of FRET partners and, on the other hand, that the excitation of the fluorescent compound at the excitation wavelength of said compound is prolonged (several seconds). Thus, the process of the present invention advantageously only utilises a single type of fluorescent compound. Several molecules of the same fluorescent compound can be used in the process according to the invention and not several molecules of at least two different fluorescent compounds as in the FRET technique.

Advantageously, the biological sample used in the context of the present invention is selected from the group consisting of a cell, several cells, a part of a cell, a cell preparation and mixtures thereof.

In the context of the present invention, “cell” is understood to mean both a cell of the prokaryotic type and of the eukaryotic type. Among the eukaryotic cells, the cell may be a yeast such as a yeast of the genus Saccharomyces or Candida, a mammalian cell, a plant cell or an insect cell. The mammalian cells can in particular be tumour cells, normal somatic line cells or stem cells. They can, non-exclusively, be red cells, osteoblasts, neurone cells, hepatocytes, muscle cells, lymphocytes or progenitor cells. The cells of the prokaryotic type are bacteria which may be gram + or −. Among these bacteria, by way of example and non-exhaustively, bacteria belonging to the division of the spirochaetes and the chlamydiae, bacteria belonging to the families of the entero-bacteria (such as Escherichia coli), streptococci (such as streptococcus), micrococci (such as staphylococcus), legionellae, mycobacteria, bacilli and others may be cited.

The cells utilised in the context of the present invention can be obtained from a primary cell culture or from a culture of a cell line or from a sample of a fluid such as water or a biological fluid previously extracted from a human or animal body, said sample possibly having undergone various previous treatments such as centrifugation, concentration, dilution . . . .

In the context of the present invention “part of a cell” is in particular understood to mean the totality or a portion of the cell membrane. In the context of the present invention, “cell membrane” is understood to mean both the phospholipid-rich plasma membrane of eukaryotic cells (also called the cyto-plasmic membrane, plasmalemma or plasmatic membrane) and the plasma membrane and the glucidic cell wall (containing peptidoglycan) of bacteria or of plant cells.

In the context of the present invention, “cell preparation” is understood to mean both a cell extract and a preparation enriched in cell organelles such as nuclei, mitochondria, Golgi apparatus, endosomes or lysosomes. Among the preferred cell extracts, the cell nucleic acids and in particular the cell DNA may be cited.

The cell parts and cell preparations utilised in the context of the present invention can be obtained from cells derived from a cell culture or from a sample of a fluid as defined above. The person skilled in the art knows various techniques making it possible to obtain, from cells or from cell cultures, cell membranes, parts of cell membranes, fractions rich in cell membranes, extracts and cell preparations involving techniques such as the phase partitioning technique or steps such as centrifugation steps.

In the context of the present invention, “fluorescent compound” is understood to mean a compound which, when it is excited at a characteristic wavelength called the excitation wavelength, absorbs a photon in this excitation range and returns to its ground state giving back a proton emitted at a wavelength which is also characteristic called the emission wavelength.

Any fluorescent compound known to the person skilled in the art can be used in the context of the present invention. Advantageously, the fluorescent compound used in the context of the present invention exhibits a favourable spectral overlap between the excitation and emission spectra. As non-restrictive examples of fluorescent compounds capable of being used in the present invention, dyes such as Alexa Fluor® 350, Alexa Fluor® 488, Alexa Fluor® 532, Alexa Fluor® 633, Alexa Fluor® 647, Alexa Fluor® 660, Alexa Fluor® 680, allophycocyanin, aminomethylcoumarin acetic acid, Cy2®, Cy 5.1®, Cy 5®, Cy 5.5 ®, dichlorofluorescein (DCFH), dihydrorhodamine (DHR), eGFP (for “enhanced GFP”), Fluo-3, FluorX®, fluorescein, fluorescein

5-maleimide, fluorescein isothiocyanate (FITC), PerCP, R-Phycoerythrin (PE), the tandem R-Phycoerythrin-Cyanine 5 or SpectralRed® or CyChrome®, R-Phyco-erythrin-Cyanine 5.5 (PE-CY 5.5®), R-Phycoerythrin-Cyanine 7 (PE-CY 7®), R-Phycoerythrin-Texas Red-x®, Red 613®, Rhodamine 110, Rhodamine 123, S65L, S65T, tetra-methylrhodamine isothiocyanate, Texas-Red-x®, TruRed®, indo 1, nano crystals (Quantum Dots), Fura 2, Fura 3, quin and DS Red may be cited.

In a preferred modification of the invention, the fluorescent compound utilized is a fluorescent compound specific to nucleic acids. “Nucleic acid” is understood to mean a single-stranded or double-stranded desoxyribonucleic acid (DNA), a ribo-nucleic acid (RNA) such as a messenger RNA or a ribosomal RNA.

The fluorescent compounds specific for nucleic acids are in particular selected from the fluorescent intercalating agents, dyes binding to the bases A:T or the bases G:C and the permeating or non-permeating cyanines. More particularly, said fluorescent compounds are selected from the group consisting of ethidium bromide, thiazole orange, thiazole blue and derivatives thereof, thioflavin S, thioflavin T, thioflavin TCN®, diethylquinolylthio-cyanine iodide (DEQTC), TOTO-l®, TO-PRO-1®, or else YOYO-1®, Hoechst® 33258, Hoechst® 33342, Hoechst® 34580, diamidino phenylindole (DAPI), propidium iodide, pyronin Y, 7-aminoactinomycin D (7 AAD), acridine orange, auramine O, calcein, New Methylene Blue, olamin-O, Oxazine 750, astra blue, SYTOX® Green, and the SYTO® series comprising in particular SYTO 11®, SYTO 12®, SYTO 13®, SYTO 15®, SYTO 16®, SYTO 18®, SYTO 62®, SYTO 80® or SYTO 81®.

In the context of the present invention, the fixing of the fluorescent compound onto said biological sample can be direct. This aspect of the invention is in particular that described in the experimental section below with the fluorescent compounds of the SYTO® series being fixed directly onto the DNA contained in the biological sample.

In one modification of the present invention, the fixing of the fluorescent compound onto said biological sample can be indirect. In this modification, the fluorescent compound is only fixed onto said biological sample via a fixing agent. In the context of the present invention, “fixing agent” is understood to mean a compound capable of being fixed onto said biological sample and onto which the fluorescent compound is fixed directly or indirectly. Advantageously, the fixing agent is selected from the group consisting of a peptide, a protein, an antibody, an agonist or antagonist of membrane or nuclear receptors, a hormone, a nucleic acid, etc. . . . The fixing of the fluorescent compound can be effected directly onto said binding agent and in particular via a covalent bond. Alternatively, this fixing can be indirect via a binding arm capable of binding the fluorescent compound to said binding agent. The person skilled in the art knows various types of binding agent and will, depending on the fluorescent compound and the binding agent utilized, know how to select the most appropriate binding agent. Likewise, the person skilled in the art knows various techniques making it possible to prepare binding agents directly or indirectly bearing a fluorescent compound. These techniques belong in particular to the field of genetic engineering and to that of chemical synthesis.

In the context of the present invention, “condition” is understood to mean both an environmental or experimental, physical or chemical condition, capable of causing changes in the biological sample and, in particular, physiological changes within that biological sample. Advantageously, the condition whose influence on a biological sample it is desired to determine is a physical or chemical condition.

“Physical condition” is understood to mean a physical condition which modifies the environment in which the biological sample is situated such as a thermal condition (modification of the temperature of said environment), an electrical condition (environment and hence biological sample subjected to an electrical stimulus) or a mechanical condition.

“Chemical condition” is understood to mean a chemical condition which modifies the environment in which the biological sample is situated such as the addition of a compound to be tested or of a sample E as defined below into the environment and/or the modification of its concentration, or the modification of the nature and/or of the concentration of the ions contained in said environment.

More particularly, the present invention relates to a process for determining the influence of a condition on a biological sample comprising the steps consisting in:

a) subjecting said biological sample to said condition;

b) placing said fluorescent compound in contact with said sample, the steps (a) and (b) being in any order;

c) exciting said fluorescent compound at the appropriate excitation wavelength for a period t the starting point whereof is t0,

d) establishing the kinetic profile of the fluorescence emitted by said fluorescent compound during the excitation period t, the fluorescence measured at t0 serving as the reference value, and

e) possibly, comparing the kinetic profile obtained in step (d) with a reference kinetic profile.

Consequently, in a first implementation of the process according to the invention, this latter comprises the steps consisting in:

a) subjecting said biological sample to said condition;

b) placing said fluorescent compound in contact with said sample;

c) exciting said fluorescent compound at the appropriate excitation wavelength for a period t the starting point whereof is t0,

d) establishing the kinetic profile of the fluorescence emitted by said fluorescent compound during the excitation period t, the fluorescence measured at t0 serving as the reference value, and

e) possibly, comparing the kinetic profile obtained in step (d) with a reference kinetic profile.

In a second implementation of the process of the invention, this latter comprises the steps consisting in:

b) placing said fluorescent compound in contact with said sample;

a) subjecting said biological sample to said condition;

c) exciting said fluorescent compound at the appropriate excitation wavelength for a period t the starting point whereof is t0,

d) establishing the kinetic profile of the fluorescence emitted by said fluorescent compound during the excitation period t, the fluorescence measured at t0 serving as the reference value, and

e) possibly, comparing the kinetic profile obtained in step (d) with a reference kinetic profile.

The steps (a) and (b) of the process according to the invention are routine steps for the person skilled in the art who will know how to implement them appropriately taking account of the type of biological sample, the type of condition and the type of fluorescent compound utilized.

For this purpose, the process of the present invention may require a supplementary permeabilization step. This supplementary step can be obligatory when the fluorescent compound cannot, by its nature, be attached to said biological sample in the absence of any permeabilization. Any permeabilization technique known to the person skilled in the art can be used in the context of the present invention. Advantageously, this permeabilization step can necessitate the use of detergents such as Triton X100.

The process of the present invention can necessitate a supplementary step of attachment of the biological sample. Any technique for attachment of a biological sample and in particular of cells known to the person skilled in the art can be used in the context of the present invention such as fixing in 70% ethanol.

The appropriate excitation wavelength used in step (c) of the process according to the invention corresponds to the characteristic excitation wavelength as defined above of the fluorescent compound used in step (b) of the process. The person skilled in the art knows the value of this wavelength or can easily obtain it without any inventive effort.

The period t during which said fluorescent compound is excited at the appropriate excitation wavelength is a long period. Advantageously, this period t lies between 1 and 1000 seconds, particularly between 10 and 800 seconds, in particular, between 20 and 500 seconds, more particularly, between 30 and 200 seconds and, quite particularly, between 40 and 100 seconds. This continued excitation can be generated by several successive excitation flashes.

Step (d) of the process consists in establishing the kinetic profile of the fluorescence emitted by said fluorescent compound during the excitation period t the starting point whereof is t0. More particularly, this step (d) consists in measuring the fluorescence emitted by the fluorescent compound at different times during the excitation period t. These different times can be selected at regular intervals or at irregular intervals. Advantageously, the measurements are made at regular intervals lying between 0.1 and 10 seconds and particularly between 1 and 5 seconds and, in particular, 2 seconds. The measured values of fluorescence emitted are then expressed relative to the value obtained at t0, this latter thus serving as a reference value. It should be noted that the fact of expressing the measured values relative to the initial value obtained at t0 makes it possible to overcome problems connected with the nature of the biological sample utilized. By way of example, particularly in the case where the biological sample is an adherent or suspension cell culture, the problems connected with the number of cells contained in said sample may be cited. In fact, with the calculation mode described above, the kinetic profile measured for a sample will be strictly identical whatever the number of cells present in the sample. It will thus be possible to compare results obtained on samples of variable size (variable number of cells).

Any instrument known to the person skilled in the art in the field of fluorescence can be utilized during the excitation of step (c) and during the measurement of the fluorescence emitted in step (d). As non-limiting examples, a fluorescence microscope equipped with a mercury lamp or a fluorescence micro-scope equipped with a xenon flash lamp may be cited.

The kinetic profile obtained thus makes it possible to assess the influence on a biological sample of the condition to which the latter is subjected. In fact, when the kinetic profile obtained exhibits values higher than the reference value at time t0, it can be concluded from this that the biological sample is in a state which allows or which has little or no effect on the homo-FRET phenomenon for the fluorescent compound utilized. By way of example and in the case of a fluorescent compound binding to double-stranded DNA, such a profile makes it possible to conclude that the condition makes it possible for the DNA contained in the biological sample to conserve or to adopt a compacted structure.

Conversely, when the kinetic profile exhibits values lower than the reference value at time t0, it can be concluded from this that the biological sample is in a state wherein no homo-FRET is possible. By way of example and in the case of a fluorescent compound binding to double-stranded DNA, such a profile makes it possible to conclude that the condition has induced degradation, reorganization and/or segmentation of the DNA contained in the biological sample.

Thus, the process of the invention can make it possible to distinguish between two cell states corresponding to two states in the structure of the DNA. For example, the invention can be applied to treatment with an apoptosis inducer, an event known to modify the structure of the DNA (reorganization, degradation and segmentation). State 1 then corresponds to cells which are untreated or are not sensitive to the inducer. An increase in the fluorescence observed under illumination is then observed. State 2 corresponds in particular to apoptotic cells (degraded DNA). No increase in fluorescence is observed under illumination.

It can however be necessary to compare the kinetic profile obtained in step (d) with a reference kinetic profile in which the state of the biological sample is completely defined. Thus, the process of the invention can comprise a supplementary and optional step (e) consisting in comparing the kinetic profile obtained in step (d) with a reference kinetic profile.

By way of example and in the case of a fluorescent compound binding to double-stranded DNA, a reference kinetic profile can be:
either the kinetic profile obtained when the biological sample is placed in the presence of a compound such as an apoptogenic agent or an anticancer agent under conditions such that the majority or even the totality of the DNA contained in the sample has been degraded, reorganized and/or segmented,
or the kinetic profile obtained when the biological sample does not undergo any treatment or stimulation capable of resulting in any degradation, reorganization or segmentation of DNA.

This step (e) of comparison of different kinetic profiles can be necessary when the condition to which the biological sample is subjected results in a state intermediate between the states 1 and 2 as previously defined.

It should be noted that in the context of the comparison of identical biological samples subjected to at least one different condition, it is not essential to have a measurement of the fluorescence emitted by the fluorescent compound at different times during the excitation period t, and that the measurement at a single given time is sufficient.

In this particular form of implementation of the invention, the present invention relates to a process for determining the influence of a condition on a biological sample comprising the steps consisting in:

a′) dividing said biological sample into a first portion A and a second portion B;

b′) subjecting said portion A to said condition;

c′) placing said fluorescent compound in contact with the portions A and B of said biological sample, the steps (b′) and (c′) being in any order;

d′) exciting said fluorescent compound at the appropriate excitation wavelength for a period t the starting point whereof is t0,

e′) measuring, for each portion of sample, the fluorescence emitted by said fluorescent compound at a time T lying within the excitation period t, the fluorescence measured at t0 for the portion A and the portion B serving as the reference value for the fluorescences measured respectively for the portion A and the portion B, and

f′) comparing the values obtained in step (e′) for the portion A and the portion B of said biological sample.

Everything which was previously described for the process according to the invention and in particular for steps (a) to (e) of the process according to the invention applies mutatis mutandis to steps (a′) to (f′) of this modification.

The time T at which the measurement of the fluorescence is performed can be any time lying within the excitation period t with the exception of the time t0. Advantageously, said time T is greater than (t0+0.5 seconds), especially greater than (t0+10 seconds) and in particular greater than (t0+15 seconds). Other measurements of the fluorescence emitted can be performed at other times T₁, T₂, etc. . . .

The comparison during step (f′) can be performed by subtracting the value obtained at the moment T for the portion B of the sample (i.e. the portion of the sample serving as the control) from the value obtained at the moment T for the portion A of the sample (i.e. the portion of the sample having been subjected to the condition). If the value obtained after said subtraction is negative, it can be concluded that the condition to which the sample was subjected induced a diminution or even disappearance of the homo-FRET phenomenon which existed in the portion B of the sample.

The present invention includes many applications particularly in the context of the screening of compounds of pharmaceutical interest. Consequently, the present invention also relates to a process for identifying a compound capable of modulating a biological process comprising a step of carrying out a process as previously defined, said test compound being the condition to which the biological sample is subjected.

In the context of the present invention, “compound capable of modulating a biological process” is understood to mean an agent capable of inhibiting, activating, accelerating or retarding said biological process. As non-limiting examples of biological processes that can be studied in the context of the present invention, apoptosis, necrosis, membrane protein rearrangement and cell division may be cited.

The term “compound” as used in the present invention refers to a molecule of any type including a chemical compound or a mixture of chemical compounds, a peptide sequence, a nucleotide sequence such as an antisense sequence, a biological macromolecule or an extract of a biological material derived from algae, bacteria, cells or tissues of animals in particular mammals, of plants or of fungi. This compound can thus be a natural compound or a synthetic compound in particular obtained by combinatorial chemistry.

The present invention also finds an application in quality control. In fact, this latter can be utilized in order to detect the presence of a compound capable of modulating a biological process in a sample E. Thus, the present invention also relates to a process for detecting the presence of a compound capable of modulating a biological process in a sample E comprising a step of carrying out a process as previously defined, said sample E being the condition to which the biological sample is subjected.

In this application, the compound capable of being present in the sample is as defined above. It may be a toxin or a mixture of toxins. The sample E can be any sample capable of undergoing quality control and in particular a natural or synthetic raw material, a natural product, a pharmaceutical product, a manufactured product, a food product, etc. . . .

In addition, the reference profile as defined above can be obtained:
from a control containing no compound capable of modulating a biological process such as a control sample E containing no compound capable of modulating a biological process, or
from a control containing a known quantity of a compound or of a mixture of compounds capable of modulating a biological process.

Likewise, the portion B of the biological sample as described above can if necessary be subjected to a control sample E containing no compound capable of modulating a biological process or containing a known quantity of a compound or of a mixture of compounds capable of modulating a biological process.

This application in quality control is of particular interest in the detection of at least one marine toxin or a mixture of marine toxins, said marine toxin or said mixture being the compound capable of modulating a biological process according to the present invention. “Marine toxin” is also understood to mean a phycotoxin and in particular a toxin selected from domoic acid, okadaic acid, the azaspiracids, the ciguatoxins, gambiertoxin, gymnodimine, the maitotoxins, palytoxin, the pectenotoxins, the spirolides and mixtures thereof. In this case, the sample E utilized can be an extract from molluscs such as oysters, mussels, clams, cockles, scallops, pectinidae, ormers and mixtures thereof. In the context of the present invention, “extract from molluscs” is understood to mean an extract obtained by grinding from whole molluscs (i.e. with shell), an extract obtained by grinding of the body of molluscs or an extract obtained by grinding of particular parts of the body of molluscs such as the digestive part or the fatty fraction of the digestive part. This extract can if necessary undergo other treatments, before being placed in contact with the biological sample, such as centrifugation, solubilisation, etc. . . .

The invention will be better understood on reading the figures and examples which follow. The purpose of these is not to limit the invention in its applications, it is merely to illustrate here the possibilities offered by the process of the invention.

### BRIEF DESCRIPTION OF DIAGRAMS

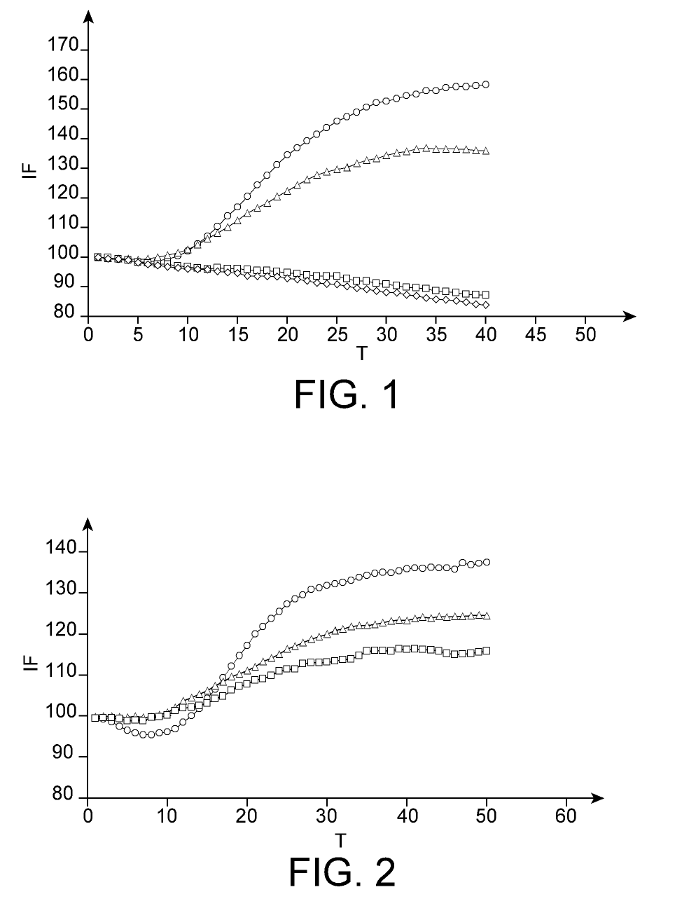

FIG. 1 shows the kinetic profile of the illumination of eukaryotic cells treated or not treated with different quantities of an apoptopic agent and in the presence of SYTO62. HeLa cells were treated for 7 hours with (Δ, □, ⋄) or without (◯) 0.1 μM (Δ), 0.5 μM (□) and 1 μM (⋄) staurosporine. The cells were then labelled with a 10 μM solution of SYTO62 then subjected to continuous illumination for 40 seconds. The intensity of fluorescence is then measured every second. Each intensity value is then expressed as a percentage of the first fluorescence value measured. (IF: intensity of fluorescence; T: illumination time).

FIG. 2 shows the kinetic profile of the illumination of eukaryotic cells treated or not treated with different quantities of an anticancer agent and in the presence of SYTO62. HeLa cells were treated for 24 hours with (Δ, □) or without (◯) 0.1 μM (Δ) and 1 μM (□) Paclitaxel (Taxol). The cells were then labelled with a 10 μM solution of SYTO62 then subjected to continuous illumination for 50 seconds. The intensity of fluorescence is then measured every second. Each intensity value is then expressed as a percentage of the first fluorescence value measured. (IF: intensity of fluorescence; T: illumination time).

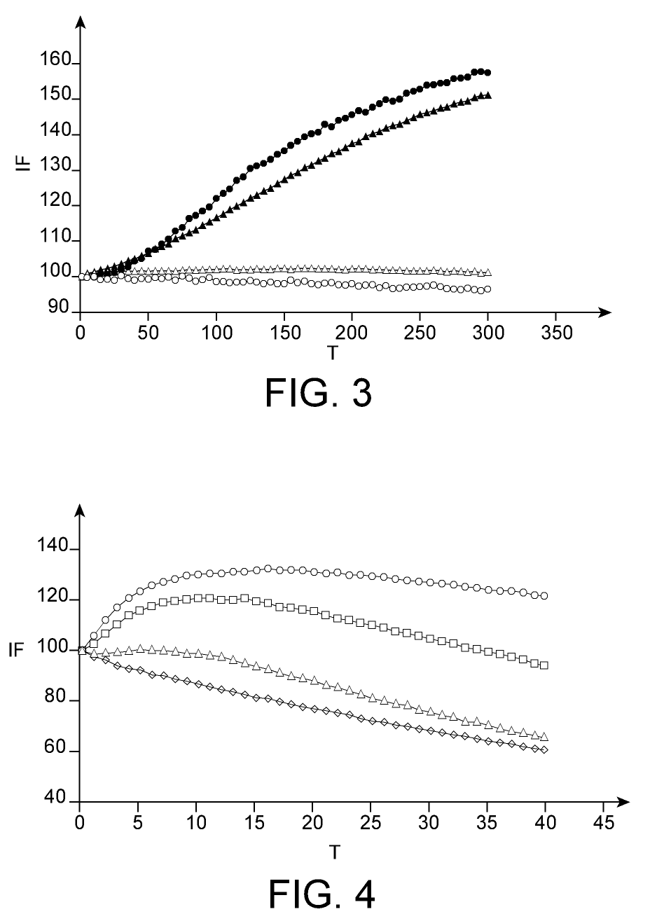

FIG. 3 shows the kinetic profile of the illumination of eukaryotic cells treated or not treated with different quantities of an apoptopic agent and in the presence of SYTO13 or SYTO15. HeLa cells were treated for 7 hours with (◯,Δ) or without (,▴) 1 μM staurosporine. The cells were then labelled with a 10 μM solution of SYTO13 (Δ,▴) or SYTO15 (◯,) then subjected to continuous illumination for 300 seconds. The intensity of fluorescence is then measured every 5 seconds. Each intensity value is then expressed as a percentage of the first fluorescence value measured. (IF: intensity of fluorescence; T: illumination time).

FIG. 4 shows the kinetic profile of the illumination of prokaryotic cells in the presence of different quantities of SYTO62. Bacteria were labelled for 30 minutes with a 6.25 μM (⋄), 12.5 μM (Δ), 25 μM (□) or 50 μM (◯) solution of SYTO62. The bacteria are then subjected to continuous illumination for 40 seconds. The intensity of fluorescence is then measured every 5 seconds. Each intensity value is then expressed as a percentage of the first fluorescence value measured. (IF: intensity of fluorescence; T: illumination time).

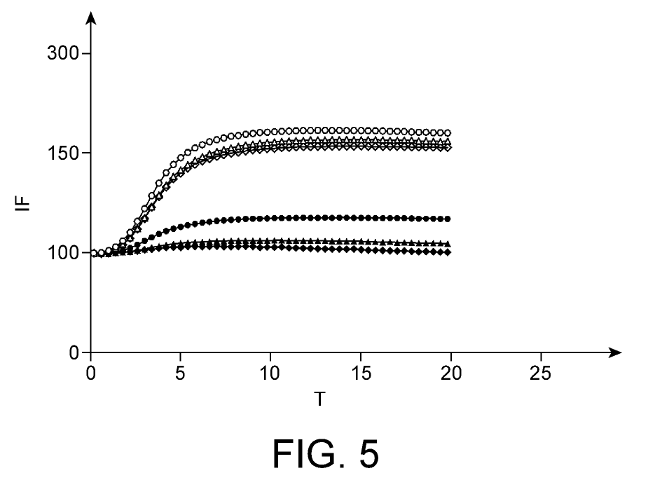

FIG. 5 shows the kinetic profile of the illumination of HepG2 cells treated for 24 hours with 0.01 μM (Δ), 0.031 μM (⋄), 0.1 μM (), 0.31 μM (▴) and 1 μM (♦) okadaic acid or without it (◯). The cells were then labelled with a solution of SYTO13 of 2 μM final concentration then subjected to continuous illumination for 20 seconds. The intensity of fluorescence is then measured every 0.4 seconds. Each intensity value is then expressed as a percentage of the first fluorescence value measured. (IF: intensity of fluorescence; T: illumination time).

### DETAILED DISCLOSURE OF PARTICULAR IMPLEMENTATION MODES

### Example 1

### Process According to the Invention Implemented on Eukaryotic Cells with Different Quantities of an Apoptopic Agent

HeLa cells (Sigma-Aldrich) are plated into a 96-well format transparent-bottomed black microplate at 20000 cells per well the day before the experiment. On the day of the test the cells are or are not treated with 0.1, 0.5 and 1 μM of staurosporine (Sigma-Aldrich) for 7 hours. The cells are then labelled for 20 minutes with 50 μl of a 10 μM solution of SYTO62 (Invitrogen) prepared in a buffer containing 25 mM Hepes, 140 mM NaCl, 1 mM EDTA and 0.1% bovine serum albumin (BSA), pH 7.4 (buffer A).

The samples are placed under a fluorescence microscope (Leica DMIRB) at ×100 magnification then subjected to continuous illumination at 488 nm with a mercury lamp (HBO 103W/2) for 40 seconds. The intensity of fluorescence emitted by the biosensor is then measured every second. The results are shown in FIG. 1.

Under the experimental conditions described, the HeLa cells not treated with the apoptotic agent staurosporine exhibit, under continuous illumination, an increase in the intensity of fluorescence measured as a function of illumination time. When the cells are treated with 0.1 μM staurosporine, a dose making it possible to induce an intermediate level of apoptosis, a weaker increase in fluorescence than in the untreated condition is measured. When the cells are treated with 0.5 μM and 1 μM staurosporine, doses making it possible to induce respectively a suboptimal and optimal level of apoptosis, an increase in fluorescence is no longer measured.

In the case of the untreated cells, the proximity between molecules of marker make it possible to establish a homoFRET mechanism due to the level of compaction of the DNA. In this condition, the illumination makes it possible to remove the fluorescence inhibition. When the cells enter apoptosis, to extents varying with the concentration of agent utilised, the entirety of the DNA is altered, being characterized by an increase in the distance between molecules of SYTO62. Under these conditions, the inhibition of fluorescence is weaker and the amplitude of the variation in fluorescence under illumination smaller (0.1 μM). At concentrations of 0.5 μm and 1 μM the distance between molecules becomes too great for the homoFRET to become established. No removal of inhibition can therefore be measured.

### Example 2

### Process According to the Invention Implemented on Eukaryotic Cells with Different Quantities of an Anticancer Agent

HeLa cells (Sigma-Aldrich) are plated into a 96-well format transparent-bottomed black microplate at 20000 cells per well the day before the experiment. On the day of the test the cells are or are not treated with 0.1 and 1 μM Paclitaxel or “Taxol” (Sigma-Aldrich) for 24 hours. The cells are then labelled for 20 minutes with 50 μl of a 10 μM solution of SYTO62 (Invitrogen) prepared in buffer A.

The samples are placed under a fluorescence microscope (Leica DMIRB) at ×100 magnification then subjected to continuous illumination at 488 nm with a mercury lamp (HBO 103W/2) for 50 seconds. The intensity of fluorescence emitted by the biosensor is then measured every second. The results are shown in FIG. 2.

Untreated HeLa cells subjected to continuous illumination exhibit a kinetic increase in the fluorescence measured. Treatment of the cells with 0.1 μM and 1 μM anticancer agent Taxol (Placlitaxel) induces a dose-dependent decrease in the variation in fluorescence measured under illumination.

### Example 3

### Process According to the Invention Implemented on Eukaryotic Cells with Different Quantities of an Apoptogenic Agent in the Presence of Different Fluorescent Compounds (SYTO13 and SYTO15)

HeLa cells (Sigma-Aldrich) are plated into a 96-well format transparent-bottomed black microplate at 20000 cells per well the day before the experiment. On the day of the test the cells are or are not treated with 1 μM of staurosporine for 7 hours. The cells are then labelled for 20 minutes with 50 μl of a 10 μM solution of SYTO13 (Invitrogen) or SYTO15 (Invitrogen) prepared in buffer A.

The labelled cells are placed under a fluorescence reader of the Varioskan type (Thermo Electron Corporation) equipped with a Xenon Flash lamp. The samples labelled with SYTO13 and SYTO15 are then subjected for 300 seconds to continuous illumination at 488 nm and 516 nm respectively. The fluorescence intensities emitted by these biosensors are then measured every 5 seconds. The results are shown in FIG. 3.

On untreated HeLa cells and those labelled with 10 μM SYTO13 or SYTO15, an increase in the fluorescence under continuous illumination is measured, as for SYTO62. When the cells are treated with 1 μM staurosporine, continuous illumination does not induce an increase in the fluorescence measured.

### Example 4

### Process According to the Invention Implemented on Prokaryotic Cells in the Presence of Different Quantities of a Fluorescent Compound (SYTO62)

Bacteria derived from waste water are plated into a tube the day before the experiment. On the day of the test 1 ml of bacterial suspension is centrifuged and the bacterial pellets taken up in 100 μl of 6.25, 12.5, 25 and 50 μM solutions of SYTO62 (Invitrogen) prepared in buffer A. The bacteria are incubated for 30 minutes at ambient temperature. The labelled bacteria are placed under a fluorescence microscope (Leica DMIRB) at ×100 magnification then subjected to continuous illumination at 488 nm with a mercury lamp (HBO 103W/2) for 40 seconds. The intensity of fluorescence emitted by the biosensor is then measured every second. The results are shown in FIG. 4.

The labelling of bacteria with a 6.25 μM concentration of SYTO62 induces under continuous illumination a decrease in the fluorescence measured in the course of time. When the bacteria are treated with 12.5 μM of marker, the fluorescent signal measured is stable then decreases starting from 10 seconds of illumination. At 25 μM then 50 μM SYTO62, an increase in the fluorescence measured, the amplitude whereof varies dose-dependently, is observed.

At excessively low concentrations of SYTO62, the distance between molecules of marker is too great, rendering the installation of an inhibition mechanism impossible. The increase in the concentration of SYTO62 utilized makes it possible to decrease this distance and to enable the installation of the homoFRET mechanism. This is thus characterized by the progressive appearance of an increase in the signal measured under illumination.

### Example 5

### Process According to the Invention Implemented on Eukaryotic Cells in the Presence of Different Quantities of Okadaic Acid

HepG2 cells (ATCC) are plated into a 96-well format transparent-bottomed black microplate at 20000 cells per well two days before the experiment. The day before the test the cells are or are not treated with 75 μl of 0.01, 0.031, 0.1, 0.31 and 1 μM solutions of okadaic acid (Sigma-Aldrich). The cells are incubated for 24 hours.

On the day of the test, the cells are labelled for 30 minutes at 37° C. with 25 μl of an 8 μM solution of SYTO13 (Invitrogen) (i.e. 2 μM final) prepared in MEM culture medium (Invitrogen).

The samples are placed under a fluorescence microscope at ×100 magnification then subjected to continuous illumination at 488 nm with a mercury lamp for 20 seconds. The intensity of fluorescence emitted by the biosensor is then measured every 0.4 seconds. The results are shown in FIG. 5.

The untreated cells HepG2 subjected to continuous illumination exhibit a kinetic increase in the fluorescence measured. Treatment of the cells with different quantities of okadaic acid induces a dose-dependent decrease in the variation in fluorescence measured under illumination.

1-16. (canceled)

17. A process for determining the influence of a condition on a biological sample, comprising:
establishing a kinetic profile of a fluorescence emitted, during excitation at a suitable excitation wavelength, of a fluorescent compound bound to said biological sample, said biological sample having been, prior to said excitation, subjected to said condition and said process not necessitating utilization of a fluorescence donor component and a different fluorescence acceptor component.

18. The process according to claim 1, further comprising:
a. subjecting said biological sample to said condition;
b. placing said fluorescent compound in contact with said sample, the steps (a) and (b) being in any order;
c. exciting said fluorescent compound at the appropriate excitation wavelength for a period t, the starting point whereof is t0;
d. establishing the kinetic profile of the fluorescence emitted by said fluorescent compound during the excitation period t, the fluorescence measured at t0 serving as the reference value; and
e. comparing the kinetic profile obtained in step (d) with a reference kinetic profile.

19. A process for determining the influence of a condition on a biological sample, comprising:
a. dividing said biological sample into a first portion A and a second portion B;
b. subjecting said portion A to the condition;
c. placing said fluorescent compound in contact with the portions A and B of the biological sample, the steps (b) and (c) being in any order;
d. exciting said fluorescent compound at an appropriate excitation wavelength for a period t, the starting point whereof is t0;
e. measuring, for each said portion of said sample, a fluorescence emitted by said fluorescent compound at a time T lying within the excitation period t, the fluorescence measured at t0 for the portion A and the portion B serving as the reference value respectively for the portion A and the portion B; and
f. comparing the values obtained in step (e) for the portion A and the portion B of said biological sample.

20. The process according to claim 17, wherein said biological sample is selected from the group consisting of a cell, several cells, a part of a cell, a cell preparation and mixtures thereof.

21. The process according to claim 17, wherein said condition is selected from physical conditions and chemical conditions.

22. The process according to claim 17, wherein said fluorescent compound is fixed onto said biological sample directly.

23. The process according to claim 17, wherein said fluorescent compound is fixed onto said biological sample indirectly by using a binding agent.

24. The process according to claim 23, wherein said binding agent is selected from the group consisting of a peptide, a protein, an antibody, an agonist or antagonist of membrane or nuclear receptors, a hormone, and a nucleic acid.

25. The process according to claim 17, wherein said fluorescent compound is a fluorescent compound specific to nucleic acids.

26. The process according to claim 17, wherein said fluorescent compound is selected from fluorescent intercalating agents, dyes binding to the bases A:T or the bases G:C, and permeant or impermeant cyanines.

27. A process for identifying a compound capable of modulating a biological process comprising a step of implementing a process as defined in claim 17, said test compound being the condition to which the biological sample is subjected.

28. The process according to claim 27, wherein said physiological process is selected from apoptosis, necrosis, membrane protein rearrangement, and cell division.

29. A process for detecting the presence of a compound capable of modulating a biological process in a sample E comprising a step of implementing a process as defined in claim 17, said sample E being the condition to which the biological sample is subjected.

30. The process according to claim 29, wherein said compound capable of modulating a biological process is a marine toxin or a mixture of marine toxins.

31. The process according to claim 29, wherein said sample E is an extract from molluscs.

32. The process according to claim 18, wherein said biological sample is selected from the group consisting of a cell, several cells, a part of a cell, a cell preparation, and mixtures thereof.

33. The process according to claim 18, wherein said condition is selected from physical conditions and chemical conditions.

34. The process according to claim 18, wherein said fluorescent compound is fixed onto said biological sample directly.

35. The process according to claim 18, wherein said fluorescent compound is fixed onto said biological sample indirectly by using a binding agent.

36. The process according to claim 35, wherein said binding agent is selected from the group consisting of a peptide, a protein, an antibody, an agonist or antagonist of membrane or nuclear receptors, a hormone, and a nucleic acid.

37. The process according to claim 18, wherein said fluorescent compound is a fluorescent compound specific to nucleic acids.

38. The process according to claim 18, wherein said fluorescent compound is selected from fluorescent intercalating agents, dyes binding to the bases A:T or the bases G:C, and permeant or impermeant cyanines.

39. The process according to claim 18, wherein the excitation period t lies between 1 and 1000 seconds.

40. A process for identifying a compound capable of modulating a biological process comprising a step of implementing a process as defined in claim 18, said test compound being the condition to which the biological sample is subjected.

41. The process according to claim 40, wherein said physiological process is selected from apoptosis, necrosis, membrane protein rearrangement, and cell division.

42. A process for detecting the presence of a compound capable of modulating a biological process in a sample E comprising a step of implementing a process as defined in claim 18, said sample E being the condition to which the biological sample is subjected.

43. The process according to claim 42, wherein said compound capable of modulating a biological process is a marine toxin or a mixture of marine toxins.

44. The process according to claim 42, wherein said sample E is an extract from molluscs.

45. The process according to claim 19, wherein said biological sample is selected from the group consisting of a cell, several cells, a part of a cell, a cell preparation, and mixtures thereof.

46. The process according to claim 19, wherein said condition is selected from physical conditions and chemical conditions.

47. The process according to claim 19, wherein said fluorescent compound is fixed onto said biological sample directly.

48. The process according to claim 19, wherein said fluorescent compound is fixed onto said biological sample indirectly by using a binding agent.

49. The process according to claim 48, wherein said binding agent is selected from the group consisting of a peptide, a protein, an antibody, an agonist or antagonist of membrane or nuclear receptors, a hormone, and a nucleic acid.

50. The process according to claim 19, wherein said fluorescent compound is a fluorescent compound specific to nucleic acids.

51. The process according to claim 19, wherein said fluorescent compound is selected from fluorescent intercalating agents, dyes binding to the bases A:T or the bases G:C, and permeant or impermeant cyanines.

52. The process according to claim 19, wherein the excitation period t lies between 1 and 1000 seconds.

53. A process for identifying a compound capable of modulating a biological process comprising a step of implementing a process as defined in claim 19, said test compound being the condition to which the biological sample is subjected.

54. The process according to claim 53, wherein said physiological process is selected from apoptosis, necrosis, membrane protein rearrangement, and cell division.

55. A process for detecting the presence of a compound capable of modulating a biological process in a sample E comprising a step of implementing a process as defined in claim 19, said sample E being the condition to which the biological sample is subjected.

56. The process according to claim 55, wherein said compound capable of modulating a biological process is a marine toxin or a mixture of marine toxins.

57. The process according to claim 55, wherein said sample E is an extract from molluscs.
